(19) United States
(12) Patent Application Publication
(10) Pub. No.: US 2011/0081388 A1
(43) Pub. Date: Apr. 7, 2011

(54) Detergent Composition

(76) Inventors: Hiroshi Oh, Cincinnati, OH (US); Valerie Jean Bradford, Waltham, MA (US); Faiz Fiesal Sherman, Mason, OH (US); Tim Bates, Cincinatti, OH (US); Vladimir Gartstein, Mason, OH (US); Ming Tang, Beijing (CN)

(21) Appl. No.: 12/899,649

(22) Filed: Oct. 7, 2010

Related U.S. Application Data
(60) Provisional application No. 61/249,279, filed on Oct. 7, 2009.

(51) Int. Cl. A61K 8/02; C11D 17/00; A61Q 11/00; A61K 8/73; A61K 8/81; A61K 8/92

(52) U.S. Cl. 424/401; 510/276; 510/218; 424/49

(57) ABSTRACT

A detergent composition having from 1% to 50% by weight of anionic surfactant and from 0.1% to 30% by weight of sudsing particles. The sudsing particles have: an average per number length of from 50 nm to 50 μm, an average per number width of from 1 nm to 500 nm, an average per number ratio length/width from 1.5 to 1000, and an hydrophobicity measured by contact angle in the range of from 30° to 100°. The weight ratio of the total amount of anionic surfactant to the total amount of sudsing particles is from 0.05 to 20.

### CROSS REFERENCE TO RELATED APPLICATIONS

This application claims the benefit of U.S. Provisional Application No. 61/249,279, filed Oct. 7, 2009.

### FIELD OF THE INVENTION

The present invention relates to detergent compositions comprising anionic surfactant and sudsing particles. The invention also concerns the use of the sudsing particles for increasing the suds volume generated by a detergent composition comprising an anionic surfactant.

### BACKGROUND OF THE INVENTION

Consumers like suds producing products for a variety of personal and cleaning uses, such as laundry detergents, hand dish washing liquids, hard surface cleaners, hair and body shampoos, facial cleansers, shave preparation gels, and oral care compositions such as dentifrices.

Consumers particularly like high and thick foams, quick foaming action, lasting foams, and the feel of rich, luxurious, creamy foams. To achieve these desirable effects, surfactants are added to many cleaning and personal care products.

Surfactants play a major role in suds producing products by lowering the dynamic surface tension of the liquid-air interface to allow gas bubbles to be formed or introduced beneath the surface of the liquid. Surfactants also stabilize the suds once it is formed.

However, high level of surfactant is not always desirable in detergent compositions. Also it is often desirable to adjust the sudsing properties of a detergent composition without reconsidering the whole formulation of the detergent composition and in particular the surfactant system. Accordingly, there remains a need for adjusting the sudsing properties of a cleaning composition with flexibility, in particular without the need to fully modify the level of surfactant used.

The inventors have discovered that some or all of the above mentioned needs could be at least partially fulfilled in the cleaning composition of the invention by using a specific ratio of anionic surfactant to specific sudsing particles.

Unless otherwise specified, all percentage and ratio are in weight.

### SUMMARY OF THE INVENTION

In one embodiment of the present invention, the invention concerns a detergent composition comprising from 0.1% to 50% by weight of anionic surfactant and from 0.1% to 30% by weight of sudsing particles,

wherein the sudsing particles have:

an average per number length in the range of from 50 nm to 50 μm,

an average per number width in the range of from 1 nm to 500 nm,

an average per number ratio length/width in the range of from 1.5 to 1000, and

an hydrophobicity measured by contact angle in the range of from 30° to 100°,

and wherein the weight ratio of the total amount of anionic surfactant to the total amount of sudsing particles is in the range of from 0.05 to 20.

The inventors have found that the combination of sudsing particles of the invention with anionic surfactant in the ratio of the invention was providing detergent composition having improved sudsing properties.

The invention also concerns the use of a sudsing particle as defined above, to increase the suds provided by a detergent composition comprising anionic surfactant.

“Hydrophobicity” as used herein shall mean the property of being water-repellent; tending to repel and not absorb water. Hydrophobicity increases with increasing contact angle.

### DETAILED DESCRIPTION OF THE INVENTION

### Anionic Surfactant

The detergent composition of the invention comprises from 0.1% to 50% by weight of at least one anionic surfactant. The detergent composition may comprise from 1% to 40% or from 3% to 30% or from 5% to 20% or from 7 to 15% of anionic surfactant.

In particular when the composition is an oral care composition, the detergent composition may comprise from 0.1% to 5%, preferably from 0.3% to 4% and most preferably from 1% to 3% of anionic surfactant.

Suitable anionic surfactants useful herein include any of the conventional anionic surfactant types typically used in detergent products.

The at least one anionic surfactant, in particular when the detergent composition is a fabric care composition, may comprise anionic surfactants selected from alkyl ester sulfonate(s); linear, branched, and modified alkylbenzene sulfonate(s); C₁₀-C₁₈ alkyl alkoxy sulfates; C₁₀₋₂₀ primary, branched-chain and random alkyl sulfates; C₁₀-C₁₈ secondary (2,3) alkyl sulfates; C₁₀-C₁₈ alkyl alkoxy carboxylate(s); fatty acid(s); mid-chain branched alkyl sulfate(s); mid-chain branched alkyl alkoxy sulfate(s); alpha-olefin sulfonate(s); phosphate ester(s); and mixtures thereof. Preferred anionic surfactants are selected from linear alkylbenzene sulfonate(s), alkoxylated or non alkoxylated alkyl sulfate(s), and mixtures thereof.

### Alkyl Ester Sulfonate Surfactant (“MES”)

As used herein, “MES” refers to alkyl ester sulfonate surfactants, commonly used in methyl ester sulfonate form. MES surfactants useful herein include sulfonated fatty acid alkyl esters of the formula R—CH(SO₃⁻)—COOR′, wherein R is, on the average, a C₆ to C₂₂ alkyl and R′ is on the average a C₁ to C₈ alkyl.

The hydrophobic portion of these sulfonated alkyl esters have the sulfonate group at the α-position, i.e., the sulfonate group is positioned at the carbon atom adjacent to the carbonyl group. The alkyl portion of the hydrophobic portion, which corresponds to the R portion of the sulfonated fatty acid alkyl esters, is on the average a C₆ to C₂₂ alkyl. Preferably, the alkyl portion of this hydrophobic portion, R, has a straight-chain of an average length C₈ to C₁₆ hydrocarbon particularly when R′ is methyl.

R′, forming the ester portion of the sulfonated alkyl esters, is on the average a C₁ to C₈ alkyl. Preferably, R′ is on the average a C₁ to C₆ alkyl, and most preferably a C₁ alkyl, i.e., methyl.

In one embodiment, the distribution is such that R is, on the average, a C₁₄ to C₁₆ alkyl (approximately, for example, a 95% C₁₄, 5% C₁₆ mixture) and R′ is methyl. In another embodiment, the distribution is such that R is, on the average, a C₁₂ to C₁₆ alkyl (approximately, for example, a 3% C₁₂, 28% C₁₄, 69% C₁₆ mixture) and R′ is methyl. In yet another embodiment, the distribution is such that R is, on the average, a C₁₀ to C₁₆ alkyl (approximately, for example, a 60% C₁₀, 35% C₁₂, 5% C₁₄ mixture) and R′ is methyl. In yet another embodiment, the distribution is such that R is, on the average, a C₁₂ to C₁₄ alkyl (approximately, for example, a 65% C₁₂, 30% C₁₄ mixture). In yet a further embodiment, blends of the aforementioned distributions of R and R′ may also be employed. In one embodiment, the methyl ester sulfonate has an average carbon length of about 16. In other embodiments, R′ could be ethyl (C₂), n-propyl & i-propyl (C₃), n-butyl, i-butyl (C₄), n-pentyl (C₅) and n-hexyl (C₆).

Methods of making alkyl ester surfactants neutralized with an alkali metal or an alkaline earth metal have been well described and are known to those skilled in art the art. See, for example, U.S. Pat. Nos. 4,671,900; 4,816,188; 5,329,030; 5,382,677; 5,384,422; 5,475,134; 5,587,500; 6,780,830. MES as such is commercially available from Huish.

### Linear, Branched, and Modified Alkylbenzene Sulfonate

The anionic surfactant may comprise linear, branched, and/or modified alkylbenzene sulfonate(s).

Exemplary anionic surfactants are C₁₀₋₁₆ alkyl benzene sulfonates, preferably C₁₁₋₁₄ alkyl benzene sulfonates. In one embodiment, the alkyl group is linear and such linear alkyl benzene sulfonates are known as “LAS”. Alkyl benzene sulfonates, and particularly LAS, are well known in the art. Such surfactants and their preparation are described for example in U.S. Pat. Nos. 2,220,099 and 2,477,383. Preferred are the linear straight chain alkylbenzene sulfonates in which the average number of carbon atoms in the alkyl group is from about 11 to 14. Particularly, C₁₁-C₁₄, e.g., C₁₂, and LAS is a specific example of such surfactants.

Other exemplary alkylbenzene sulfonates include modified alkylbenzene sulfonate (MLAS) as discussed in WO 99/05243, WO 99/05242, WO 99/05244, WO 99/05082, WO 99/05084, WO 99/05241, WO 99/07656, WO 00/23549, and WO 00/23548.

MLAS may comprise a mixture, preferably consisting essentially of: (a) from about 15% to about 99%, preferably from about 15% to about 60%, more preferably from about 20% to about 40%, by weight of a mixture of branched alkylbenzene sulfonates having formula:

L(R¹)(R²)-A-SO₃⁻

wherein L is an acyclic aliphatic moiety consisting of carbon and hydrogen, the L having two methyl termini and the L having no substituents other than A, R¹ and R²; and wherein the mixture of branched alkylbenzene sulfonates contains two or more, preferably at least three, optionally more, of the branched alkylbenzene sulfonates differing in molecular weight of the anion of the formula (I), and wherein the mixture of branched alkylbenzene sulfonates has a sum of carbon atoms in R¹, L and R² of from 9 to 15, preferably from 10 to 14; an average aliphatic carbon content, i.e., based on R¹, L and R² and excluding A, of from about 10.0 to about 14.0, preferably from about 11.0 to about 13.0, more preferably from about 11.5 to about 12.5, carbon atoms; R¹ is C₁-C₃ alkyl, preferably C₁-C₂ alkyl, more preferably methyl; R² is selected from H and C₁-C₃ alkyl, preferably H and C₁-C₂ alkyl, more preferably H and methyl, more preferably H and methyl provided that in at least about 0.5, more preferably 0.7, more preferably 0.9 to 1.0 mole fraction of the branched alkylbenzene sulfonates, R² is H; A is a benzene moiety, typically A is the moiety-C₆H₄—, with the SO₃⁻ moiety of Formula (I) in para-position to the L moiety, though in some proportion, usually no more than about 5%, preferably from 0 to 5% by weight, the SO₃⁻ moiety is ortho-to L; and (b) from about 1% to about 85%, preferably from about 40% to about 85%, more preferably from about 60% to about 80%, by weight of a mixture of nonbranched alkylbenzene sulfonates having formula:

Y-A-SO₃⁻

wherein A is as defined hereinbefore and Y is an unsubstituted linear aliphatic moiety consisting of carbon and hydrogen having two methyl termini, and wherein the Y has a sum of carbon atoms of from 9 to 15, preferably from 10 to 14, and the Y has an average aliphatic carbon content of from about 10.0 to about 14.0, preferably from about 11.0 to about 13.0, more preferably 11.5 to 12.5 carbon atoms; and wherein the modified alkylbenzene sulfonate surfactant mixture is further characterized by a 2/3-phenyl index of from about 160 to about 275, preferably from about 170 to about 265, more preferably from about 180 to about 255; and also preferably wherein the modified alkylbenzene sulfonate surfactant mixture has a 2-methyl-2-phenyl index of less than about 0.3, preferably less than about 0.2, more preferably less than about 0.1, more preferably still, from 0 to 0.05.

### C₁₀-C₁₈ Alkyl Alkoxy Sulfates

Another exemplary type of anionic surfactant includes ethoxylated alkyl sulfate surfactants. Such materials, also known as alkyl ether sulfates or alkyl polyethoxylate sulfates, are those which correspond to the formula:

R′—O—(C₂H₄O)ₙ—SO₃⁻

wherein R′ is a C₈-C₂₀ alkyl group and n is from about 1 to 20. In a specific embodiment, R′ is C₁₀-C₁₈ alkyl and n is from about 0.1 to 15. In another embodiment, n is from about 1 to 15. In more specific embodiments, R′ is a C₁₂-C₁₆, n is from about 1 to 6. In the disclosure herein, the designation “EOx” indicates that the alkoxy group is an ethoxy group, the integer “x” indicates the number of ethoxy groups in each chain.

The alkyl ether sulfates will generally be used in the form of mixtures comprising varying R′ chain lengths and varying degrees of ethoxylation. Frequently, though the average n value may be more than zero, such mixtures will inevitably also contain some non-ethoxylated alkyl sulfate materials, i.e., individual surfactant molecules of the above ethoxylated alkyl sulfate formula wherein n=0 for that particular molecule.

### C₁₀₋₂₀ Primary, Branched-Chain and Random Alkyl Sulfates

Alkyl sulfates may also be added separately to the compositions of this invention and used as or in any anionic surfactant component which may be present. Specific examples of alkyl sulfates surfactants are those produced by the sulfation of higher C₁₀-C₂₀ fatty alcohols. Conventional primary alkyl sulfate surfactants have the general formula:

ROSO₃⁻

wherein R is typically a linear C₁₀-C₂₀ alkyl group, which may be straight chain or branched chain. In specific embodiments, R is a C₁₀-C₁₅ alkyl, more specifically R is C₁₂-C₁₄.

### C₁₀-C₁₈ Secondary (2,3) Alkyl Sulfates

Another anionic surfactant useful herein includes secondary (2,3) alkyl sulfates having formulae CH₃—(CH₂)ₓ—CH(OSO₃⁻)—CH₃ or CH₃—(CH₂)_{y}—CH(OSO₃⁻)—CH₂—CH₃.

Non-limiting examples of a preferred secondary alkyl sulfate include the one where x is at least about 7, preferably at least about 9, and y is an integer of at least 8, preferably at least about 9.

### C₁₀-C₁₈ Alkyl Alkoxy Carboxylates

Another exemplary type of anionic surfactant includes ethoxylated alkyl carboxylate surfactants. Such materials, also known as alkyl ether carboxylates or alkyl polyethoxylate carboxylates, are those which correspond to the formula:

R′—O—(C₂H₄O)ₙ—COO⁻

wherein R′ is a C₈-C₂₀ alkyl group and n is an integer from about 1 to 20. In a specific embodiment, R′ is C₁₀-C₁₈ alkyl and/or n is from about 1 to 15. In more specific embodiments, R′ is a C₁₂-C₁₆ and/or n is from about 1 to 6.

The alkyl ether carboxylates will generally be used in the form of mixtures comprising varying R′ chain lengths and varying degrees of ethoxylation. Frequently such mixtures will inevitably also contain some non-ethoxylated alkyl carboxylate materials, i.e., surfactants of the above alkyl ether carboxylate formula wherein n=0.

### Fatty Acids

Fatty acids have the general formula:

RCOO⁻

wherein R is typically a C₉-C₂₁ alkyl group, which may be straight chain or branched chain. In specific embodiments, R is a C₉-C₁₇ alkyl, and more specifically R is C₁₁-C₁₅.

Exemplary fatty acids are selected from the group consisting of lauric acid, tridecylic acid, myristic acid, pentadecylic acid, palmitic acid, margaric acid, stearic acid, arachidic acid, phytanic acid, behenic acid, palmitoleic acid, oleic acid, elaidic acid, vaccenic acid, linoleic acid, cis-eleostearic acid, trans-eleosteric acid, linolenic acid, arachidonic acid and combinations thereof. Preferred fatty acids can be saturated or unsaturated. Unsaturated fatty acids typically having an iodine value from 15 to 25, preferably from 18 to 22 and a cis:trans isomer ratio from 1:1 to 200:1, preferably fro 10:1 to 200:1.

Preferred sources of fatty acid are selected from the group consisting of coconut, soybean, tallow, palm, palm kernel, rapeseed, lard, sunflower, corn, safflower, canola, olive, peanut and combinations thereof.

### Mid-Chain Branched Alkyl Sulfates

Exemplary anionic surfactants include mid-chain branched alkyl sulfates as discussed in U.S. Pat. Nos. 6,020,303 and 6,060,443.

Mid-chain branched alkyl sulfates may comprise at least about 0.5%, preferably at least about 5%, more preferably at least about 10%, most preferably at least about 20%, by weight of longer alkyl chain, mid-chain branched surfactant compounds of the formula:

A^{b}-X—B

wherein:

(a) A^{b} is a hydrophobic C₉ to C₂₂ (total carbons in the moiety), preferably from about C₁₂ to about C₁₈, mid-chain branched alkyl moiety having: (1) a longest linear carbon chain attached to the —X—B moiety in the range of from 8 to 21 carbon atoms; (2) one or more C₁₋₃ alkyl moieties branching from this longest linear carbon chain; (3) at least one of the branching alkyl moieties is attached directly to a carbon of the longest linear carbon chain at a position within the range of position 2 carbon (counting from carbon #1 which is attached to the —X—B moiety) to position ω-2 carbon (the terminal carbon minus 2 carbons, i.e., the third carbon from the end of the longest linear carbon chain); and (4) the surfactant composition has an average total number of carbon atoms in the A^{b}-X moiety in the above formula within the range of greater than 14.5 to about 17.5 (preferably from about 15 to about 17);

(b) B is a hydrophilic moiety selected from sulfates; and

(c) X is selected from —CH₂— and —C(O)—.

Also preferred are mid-chain branched alkyl sulfates of the above formula wherein the A^{b} moiety does not have any quaternary substituted carbon atoms (i.e., 4 carbon atoms directly attached to one carbon atom).

Preferred mid-chain branched alkyl sulfates herein comprise longer alkyl chain, mid-chain branched surfactant compounds of the above formula wherein the A^{b} moiety is a branched primary alkyl moiety having the formula:

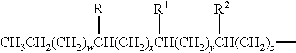

wherein the total number of carbon atoms in the branched primary alkyl moiety of this formula (Including the R, R¹, and R² branching) is from 13 to 19; R, R1, and R2 are each independently selected from hydrogen and C₁-C₃ alkyl (preferably methyl), provided R, R¹, and R² are not all hydrogen and, when z is 0, at least R or R¹ is not hydrogen; w is from 0 to 13; x is from 0 to 13; y is from 0 to 13; z is from 0 to 13; and w+x+y+z is from 7 to 13.

Also preferred mid-chain branched alkyl sulfates comprise longer alkyl chain, mid-chain branched surfactant compounds of the above formula wherein the A^{b} moiety is a branched primary alkyl moiety having the formula selected from:

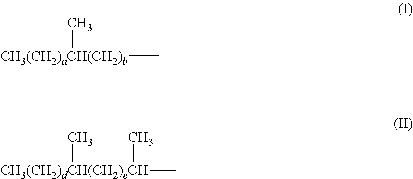

or mixtures thereof; wherein a, b, d, and e are integers, a+b is from 10 to 16, d+e is from 8 to 14 and wherein further when a+b=10, a is an integer from 2 to 9 and b is an integer from 1 to 8; when a+b=11, a is an integer from 2 to 10 and b is an integer from 1 to 9; when a+b=12, a is an integer from 2 to 11 and b is an integer from 1 to 10; when a+b=13, a is an integer from 2 to 12 and b is an integer from 1 to 11; when a+b=14, a is an integer from 2 to 13 and b is an integer from 1 to 12; when a+b=15, a is an integer from 2 to 14 and b is an integer from 1 to 13; when a+b=16, a is an integer from 2 to 15 and b is an integer from 1 to 14; when d+e=8, d is an integer from 2 to 7 and e is an integer from 1 to 6; when d+e=9, d is an integer from 2 to 8 and e is an integer from 1 to 7; when d+e=10, d is an integer from 2 to 9 and e is an integer from 1 to 8; when d+e=11, d is an integer from 2 to 10 and e is an integer from 1 to 9; when d+e=12, d is an integer from 2 to 11 and e is an integer from 1 to 10; when d+e=13, d is an integer from 2 to 12 and e is an integer from 1 to 11; when d+e=14, d is an integer from 2 to 13 and e is an integer from 1 to 12.

### Mid-Chain Branched Alkyl Alkoxy Sulfates

Still other exemplary anionic surfactants include mid-chain branched alkyl alkoxy sulfates as discussed in U.S. Pat. Nos. 6,008,181 and 6,020.303

Mid-chain branched alkyl alkoxy sulfates comprise from about 0.001% to about 100% of one or more (preferably a mixture of two or more) mid-chain branched primary alkyl alkoxylated sulfates having the formula:

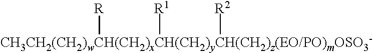

wherein the total number of carbon atoms in the branched primary alkyl moiety of this formula (Including the R, R¹, and R² branching, but not including the carbon atoms in the EO/PO alkoxy moiety) is from 14 to 20, and wherein further for this surfactant mixture the average total number of carbon atoms in the branched primary alkyl moieties having the above formula is within the range of greater than 14.5 to about 17.5 (preferably from about 15 to about 17); R, R¹, and R² are each independently selected from hydrogen and C₁-C₃ alkyl (preferably methyl), provided R, R¹, and R² are not all hydrogen and, when z is 1, at least R or R¹ is not hydrogen; w is an integer from 0 to 13; x is an integer from 0 to 13; y is an integer from 0 to 13; z is an integer of at least 1; w+x+y+z is from 8 to 14; and EO/PO are alkoxy moieties including for example ethoxy, propoxy, butoxy, etc, preferably selected from ethoxy, propoxy, and mixed ethoxy/propoxy groups, most preferably ethoxy, wherein m is at least about 0.01, preferably within the range of from about 0.1 to about 30, more preferably from about 0.5 to about 10, and most preferably from about 1 to about 5. It is to be recognized that the (EO/PO)ₘ moiety may be either a distribution with average degree of alkoxylation corresponding to m, or it may be a single specific chain with alkoxylation (e.g., ethoxylation and/or propoxylation) of exactly the number of units corresponding to m.

Preferably, the mid-chain branched alkyl alkoxy sulfates comprise a mixture of mid-chain branched primary alkyl alkoxylated sulfate surfactants, said mixture comprising at least about 5% by weight of two or more mid-chain branched primary alkyl alkoxylated sulfates having the formula:

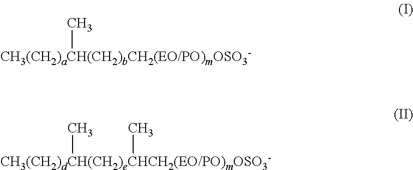

or mixtures thereof; wherein a, b, d, and e are integers, a+b is from 10 to 16, d+e is from 8 to 14 and wherein further when a+b=10, a is an integer from 2 to 9 and b is an integer from 1 to 8; when a+b=11, a is an integer from 2 to 10 and b is an integer from 1 to 9; when a+b=12, a is an integer from 2 to 11 and b is an integer from 1 to 10; when a+b=13, a is an integer from 2 to 12 and b is an integer from 1 to 11; when a+b=14, a is an integer from 2 to 13 and b is an integer from 1 to 12; when a+b=15, a is an integer from 2 to 14 and b is an integer from 1 to 13; when a+b=16, a is an integer from 2 to 15 and b is an integer from 1 to 14; when d+e=8, d is an integer from 2 to 7 and e is an integer from 1 to 6; when d+e=9, d is an integer from 2 to 8 and e is an integer from 1 to 7; when d+e=10, d is an integer from 2 to 9 and e is an integer from 1 to 8; when d+e=11, d is an integer from 2 to 8 and e is an integer from 1 to 9; when d+e=12, d is an integer from 2 to 11 and e is an integer from 1 to 10; when d+e=13, d is an integer from 2 to 12 and e is an integer from 1 to 11; when d+e=14, d is an integer from 2 to 13 and e is an integer from 1 to 12; wherein for this surfactant mixture the average total number of carbon atoms in the branched primary alkyl moieties having the above formulas is within the range of greater than 14.5 to about 17.5; and wherein EO/PO are alkoxy moieties, preferably selected from ethoxy, propoxy, and mixed ethoxy/propoxy groups, wherein m is at least about 0.01, preferably within the range of from about 0.1 to about 30, more preferably from about 0.5 to about 10, and most preferably from about 1 to about 5.

The mid-chain branched alkyl alkoxy sulfates may comprise compounds of formula:

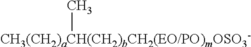

wherein: a is an integer from 2 to 11, b is an integer from 1 to 10, and a+b is 8 or 9; and EO/PO are alkoxy moieties, preferably selected from ethoxy, propoxy, and mixed ethoxy/propoxy groups, wherein m is at least about 0.01, preferably within the range of from about 0.1 to about 30, more preferably from about 0.5 to about 10, and most preferably from about 0.6 to about 5.

Also preferred herein are alkoxylated sulfate compounds of formula:

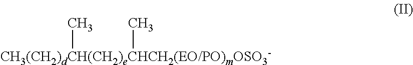

wherein: d and e are integers and d+e is 6 or 7; and wherein further when d+e=6, d is an integer from 2 to 5 and e is an integer from 1 to 4; when d+e=7, d is an integer from 2 to 6 and e is an integer from 1 to 5; and EO/PO are alkoxy moieties, preferably selected from ethoxy, propoxy, and mixed ethoxy/propoxy groups, wherein m is at least about 0.01, preferably within the range of from about 0.1 to about 30, more preferably from about 0.5 to about 10, and most preferably from about 0.6 to about 5.

### Alpha-Olefin Sulfonate

Other anionic surfactants useful in embodiments of the present disclosure include olefin sulfonates, which are compounds produced by the sulfonation of alpha-olefin by means of uncomplexed sulfur trioxide followed by neutralization of the acid reaction mixture under conditions such that sultones formed in the reaction are hydrolyzed to give corresponding hydroxyalkanesulfonates. The alpha-olefins from which the olefin sulfonates are derived are mono-olefins having from about 8 to about 24 carbon atoms, preferably from about 12 to about 16 carbon atoms. Preferably, they are straight chain olefins. Exemplary alpha-olefin sulfonates for use in the disclosure herein have the general formula:

R—CH═CH—CH₂—SO₃⁻(2,3-alkenylsulfonate) or R—CH(OH)—CH₂—CH₂—SO₃⁻(3-hydroxy-alkanesulfonate,

where R is a linear or branched alkyl of about 8 to 20 carbon atoms. Examples of suitable alpha-olefins include 1-olefins such as 1-dodecene, 1-tetradecene, 1-hexadecene, 1-octadecene, 1-eicosene and 1-tetracosene.

### Phosphate Esters

Other anionic surfactants useful in embodiments of the present disclosure include phosphate esters. Phosphate esters are any materials of the general formula:

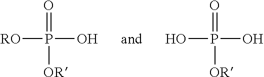

wherein R and R′ are C₆-C₂₀ alkyl or ethoxylated alkyl groups. Preferably R and R′ are of the general formula:

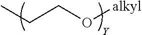

wherein the alkyl substituent is C₁₀-C₁₆ and Y is between 0 and about 4. Most preferably the alkyl substituant of that formula is C₁₀-C₁₆ and Y is between about 2 and about 4. Such compounds may be prepared by known methods from phosphorus pentoxide, phosphoric acid, or phosphorus oxy halide and alcohols or ethoxylated alcohols.

It will be appreciated that the formula depicted represent mono- and di-esters, and commercial phosphate esters will generally comprise mixtures of the mono- and di-esters, together with some proportion of tri-ester.

### Other Anionic Surfactants

In particular when the detergent composition is an oral care composition, the anionic surfactant may comprise surfactant selected from the group consisting of sarcosinate surfactants, isethionate surfactants, taurate surfactants, and mixture thereof. Preferred for use herein are alkali metal or ammonium salts of these surfactants. Most preferred herein are the sodium and potassium salts of the following: lauroyl sarcosinate, myristoyl sarcosinate, palmitoyl sarcosinate, stearoyl sarcosinate and oleoyl sarcosinate. In oral care composition, preferred anionic surfactants useful herein include the water-soluble salts of alkyl sulfates having from 10 to 18 carbon atoms in the alkyl radical and the water-soluble salts of sulfonated monoglycerides of fatty acids having from 10 to 18 carbon atoms. Sodium lauryl sulfate and sodium coconut monoglyceride sulfonates are examples of anionic surfactants of this type. Mixtures of anionic surfactants can also be utilized.

### Sudsing Particles

The detergent composition comprises from 0.1% to 30% by weight of sudsing particles. The detergent composition may comprise from 0.3% to 20%, from 0.5% to 10% or from 0.8% to 5% or from 1% to 4% or from 1.2% to 3% of sudsing particles.

The sudsing particles have, as raw material, a primary length, a primary width, and a primary ratio length/width. The sudsing particles may be in the detergent composition in the form of aggregates. The aggregates may have an average size, per weight, of less than 500 μm. The average size of the aggregate may be measured by dynamic light scattering (DLS). In the detergent composition, preferably less than 50% or 35% or 20% by weight of the sudsing particles are in the form aggregates having a size of more than 500 μm. Preferably less than 50% or 35% or 20% by weight of the sudsing particles are in the form aggregates having a size of more than 300 μm or 100 μm.

Typically, if the detergent composition comprises aggregates of sudsing particles, the aggregate will disaggregate during the washing process. This disaggregation is facilitated by the presence of surfactant and dispersant.

In the present description and claims, the term length, width, and ratio length/width of the sudsing particle refers to the primary length, the primary width, and the primary ratio length/width of the sudsing particle, even when the sudsing particle is part of an aggregate of sudsing particles.

The sudsing particles have an average per number length in the range of from 50 nm to 50 μm. In particular, the sudsing particles have an average per number length in the range of from 100 nm to 10 μm, or from 200 nm to 1 μm or from 300 nm to 900 nm or from 400 nm to 800 nm.

The sudsing particles have an average per number width in the range of from 1 nm to 500 nm. In particular, the sudsing particles have an average per number length in the range of from 2 nm to 500 nm, or from 4 nm to 250 nm, or from 8 nm to 125 nm, or from 16 nm to 60 nm.

The sudsing particles have an average per number ratio length/width in the range of from 1.5 to 1000. In particular, the sudsing particles have an average per number ratio length/width in the range of from 3 to 500, or from 6 to 250, or from 12 to 125, or from 24 to 60.

The sudsing particles have a hydrophobicity measured by contact angle in the range of from 30° to 100°. The sudsing particles may have a hydrophobicity measured by contact angle in the range of from 40° to 95°, or from 50°, or 60°, or 70° to 80° or 90°.

In the detergent composition, the weight ratio of the total amount of anionic surfactant to the total amount of sudsing particles is in the range of from 0.05 to 20. In the detergent composition of the invention, the weight ratio of the total amount of anionic surfactant to the total amount of sudsing particles may be in the range of from 0.1 to 10 or from 0.2 to 5 or from 0.5 to 2.

Preferably, the sudsing particles comprise polysaccharide, fatty acids, polyethylene, polypropylene or a mixture thereof. Preferably, the sudsing particles comprise cellulose, starch, lignin and mixture thereof. Preferably, the particles comprise cellulose.

The sudsing particles may comprise at least 20% or at least 50% or at least 80%, by weight of the particle of a polymer having a cellulosic backbone. Such polymers include Arbocel MF 40-100® from JRS company, which have a contact angle 37°; and Microcrystalline Cellulose Avicel® PH-101 from FMC Biopolymers company, which have a contact angle of 10°. Those polymers can be hydrophobically modified to increase their contact angle.

The sudsing particles may comprise at least 20% or at least 50% or at least 80%, by weight of the particle of a polymer having a lignin backbone.

The sudsing particles may comprise at least 20% or at least 50% or at least 80%, by weight of the particle of a polymer having a starch backbone. U.S. Pat. No. 6,677,386 discloses processes for producing biopolymer nanoparticles, such as starch nanoparticles.

The sudsing particles may comprise at least 20% or at least 50% or at least 80%, by weight of the particle of fatty acid(s). the fatty acid sudsing particles may be prepared by emulsification-evaporation method or emulsification-diffusion method. Such preparation is disclosed by W. Dong, R. Bodmeier, Int. J. Pharm. 326 (2006), 128-138.

The sudsing particles may comprise at least 20%, or at least 50% or at least 80%, by weight of the particle of a polymer having a polyethylene or polypropylene backbone. These particles can be prepared using Cryo-Grind® system from Air Product company.

The contact angle, as used herein means the contact angle at the particle/air/liquid interface. This can be measured using the gel trapping technique described by Paunov (Langmuir, 2003, Vol. 19, pp. 7970-7976) or by means of a commercially available goniometer (Rame-Hart, Inc. Model: 250-00-115) for measuring contact angle.

In particular, the contact angle of the sudsing particles may be measured as follow. The sudsing particles are compressed into thin disk to create a flat, homogenous, and smooth surface, using a hand held pellet press (5,000 psi). Sessile drop with water can be used to determine contact angle (CA) of the powders. The water rapidly disperses into the porous packed powder; therefore, images of the drop are preferably obtained using high speed (17 msec) video capture to minimize the effect of porosity. Contact angles may be determined via analyzing video images. The system may use a FTA 200 made by First Ten Angstroms, Inc.

The length and width of a sudsing particle may be measure as follow. A 100 ppm aqueous dispersion of the particles is prepared in 150 ppm Linear Alkyl Benzene Sulfonate solution. The samples are placed into a gold planchette and plunge frozen in liquid ethane. The specimen are then fractured at −193° C. in a Gatan Alto 2500 cryo-prep chamber, cooled back down below −160° C. and coated for 90 seconds with gold/palladium. The sample is then transferred to the Hitachi S-5200 SEM/STEM maintaining the specimen below −170° C. during the entire process. Imaging was performed at 3 KV and 5 μA tip current. Width and length of the particles are measured by the Cryo SEM (scanning electronic microscopy). The measurement of length and width and the ratio length/width is averaged on, for example, 50 particles.

The length can be defined as the distance between the two further apart points of the particle. The width can be defined as the distance between the two further apart points of the particles, on an axis perpendicular to the length. The height can be defined as the distance between the two further apart points of the particles, on an axis perpendicular to the length and the width.

Preferably, the width/height ratio is between 1 and 5, preferably between 1 and 3, preferably between 1 and 2 or between 1 and 1.5. The more “needle-like” the particle, the better it is believed to be for purposes of the invention.

The composition may comprise a sudsing particle comprising switchable functional groups attached to the surface of the particles. The switchable functional groups are switchable upon exposure to a predetermined change in one or both of pH and temperature. The switchable functional groups are sensitive to one or both of pH and temperature changes and are easily protonated or deprotonated. The switchable functional groups may be Brønsted acids or bases on the surface of the particles. Alternatively, the switchable functional groups, such as carboxyl or amino groups, may be on compositions, such as polymers, grafted onto the surface of the particles.

It may be desirable that the suds produced during a cleaning process using the detergent composition of the invention is easily rinsable. A method for reducing the level of suds during a rinsing step may be provided. The sudsing particles may be modified to include switchable functional groups. As stated above, the switchable functional groups are switchable upon exposure to a predetermined change in one or both of pH and temperature. The method for reducing the level of suds in a rinse solution includes defoaming the solution by one or more of the steps selected from (1) increasing the concentration of particles relative to surfactant and decreasing one of the pH or the temperature of the solution, and (2) increasing the size of the particles by aggregation of particles, (3) wherein the step comprises reducing available anionic surfactants by forming one of a complex and (4) wherein the step comprises reducing available anionic surfactants by forming a coacervate with positively charged particles by decreasing one of the pH or temperature of the rinse solution.

Defoaming by increasing the concentration of particles relative to surfactant may be done by adding a particulate powder, such as silica powder, to the product. Defoaming by increasing the concentration of particles relative to surfactant may be done by adding hydrophobic particles, such as milled and surface modified hydrophobic mica such as polydimethyl siloxane grafted mica, to the product at a basic pH, such as pH 10. Defoaming by increasing the size of the particles may be done by adding particles having a high aspect ratio, such as fibers and platelets. Defoaming by decreasing the pH will occur, for example, under rinse conditions wherein the pH of the rinse solution is lower than the pH of the wash solution. This is particularly applicable to both liquid and granular forms of laundry detergents, dish washing detergents, or toothpaste wherein a polyacid functional group has been grafted onto the particles. The wash solution is typically alkaline, having a pH greater than 7. A rinse solution typically has a lower pH, such as the pH of tap water, about 7 and below. Protonation of the functional group, for example by grafting a polyacrylic acid that has pKa lower than 7, upon exposure to the lower pH in a rinse solution will dramatically increase the hydrophobicity of the particles that result in forming an antifoaming aggregate.

Examples of polymers that can be used as the switchable functional groups that are switchable upon exposure to a predetermined change in temperature may be, for example, polymers, which become insoluble (in other words hydrophobic) upon heating, have a so-called lower critical solution temperature (LCST). Polymers, which become soluble (in other words hydrophilic) upon heating, have an upper critical solution temperature (UCST). Typical LCST polymers are based on N-isopropylacrylamide (NIPAM), N,N-diethylacrylamide (DEAM), methylvinylether (MVE), and N-vinylcaprolactam (NVCl). A typical UCST system is based on a combination of acrylamide (AAm) and acrylic acid (AAc), and PEO-b-PPO, PEO-b-PPO-b-PEO and PEG-b-PLGA-b-PEG block copolymers. For example, an aqueous poly(NiPAAM) solution precipitates (i.e. becomes hydrophobic) above 32° C. (LCST) and the transition is very sharp.

### Adjunct Ingredients

The detergent composition may comprise one or more adjunct ingredient(s). The precise nature of these additional adjunct components, and levels of incorporation thereof, will depend on the physical form of the composition and the nature of the operation for which it is to be used.

For example when the detergent composition is a fabric care composition, suitable adjunct materials include, but are not limited to surfactant, builder, flocculating aid, chelating agents, dye transfer inhibitors, enzymes, enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids, and/or pigments. In addition to the disclosure below, suitable examples of such other adjuncts and levels of use are found in U.S. Pat. Nos. 5,576,282, 6,306,812 B1 and 6,326,348 B1.

The detergent composition may also comprise, in particular when the detergent composition is an oral care composition, anticalculus agent, fluoride source, thickening agents, flavoring and sweetening agents, and/or antimicrobial agents.

The detergent composition may comprise a non-ionic surfactant. Where present the non-ionic surfactant(s) is generally present in amounts of from 0.01 wt % to 20 wt %, or from 0.1 wt % to 4 wt % by weight of the detergent composition.

The non-ionic surfactant can be selected from the group consisting of: alkyl polyglucoside and/or an alkyl alkoxylated alcohol; C₁₂-C₁₈ alkyl ethoxylates, such as, NEODOL® non-ionic surfactants from Shell; C₆-C₁₂ alkyl phenol alkoxylates wherein the alkoxylate units are ethyleneoxy units, propyleneoxy units or a mixture thereof; C₁₂-C₁₈ alcohol and C₆-C₁₂ alkyl phenol condensates with ethylene oxide/propylene oxide block polymers such as Pluronic® from BASF; C₁₄-C₂₂ mid-chain branched alcohols, BA, as described in more detail in U.S. Pat. No. 6,150,322; C₁₄-C₂₂ mid-chain branched alkyl alkoxylates, BAEx, wherein x=from 1 to 30, as described in more detail in U.S. Pat. No. 6,153,577, U.S. Pat. No. 6,020,303 and U.S. Pat. No. 6,093,856; alkylpolysaccharides as described in more detail in U.S. Pat. No. 4,565,647, specifically alkylpolyglycosides as described in more detail in U.S. Pat. No. 4,483,780 and U.S. Pat. No. 4,483,779; polyhydroxy fatty acid amides as described in more detail in U.S. Pat. No. 5,332,528, WO 92/06162, WO 93/19146, WO 93/19038, and WO 94/09099; ether capped poly(oxyalkylated) alcohol surfactants as described in more detail in U.S. Pat. No. 6,482,994 and WO 01/42408; and mixtures thereof.

The detergent composition may comprise a cationic surfactant. When present, preferably the detergent composition comprises from 0.01 wt % to 10 wt %, or from 0.1 wt % to 2 wt % cationic detersive surfactant.

Suitable cationic detersive surfactants are alkyl pyridinium compounds, alkyl quaternary ammonium compounds, alkyl quaternary phosphonium compounds, and alkyl ternary sulphonium compounds. The cationic detersive surfactant can be selected from the group consisting of: alkoxylate quaternary ammonium (AQA) surfactants as described in more detail in U.S. Pat. No. 6,136,769; dimethyl hydroxyethyl quaternary ammonium surfactants as described in more detail in U.S. Pat. No. 6,004,922; polyamine cationic surfactants as described in more detail in WO 98/35002, WO 98/35003, WO 98/35004, WO 98/35005, and WO 98/35006; cationic ester surfactants as described in more detail in U.S. Pat. No. 4,228,042, U.S. Pat. No. 4,239,660, U.S. Pat. No. 4,260,529 and U.S. Pat. No. 6,022,844; amino surfactants as described in more detail in U.S. Pat. No. 6,221,825 and WO 00/47708, specifically amido propyldimethyl amine; and mixtures thereof.

Cationic surfactants may be chosen among mono-C₈₋₁₀ alkyl mono-hydroxyethyl di-methyl quaternary ammonium chloride, mono-C₁₀₋₁₂ alkyl mono-hydroxyethyl di-methyl quaternary ammonium chloride and mono-C₁₀ alkyl mono-hydroxyethyl di-methyl quaternary ammonium chloride. Cationic surfactants such as Praepagen HY (tradename Clariant) may be useful and may also be useful as a suds booster.

The detergent composition of the invention may comprise a builder. When a builder is used, the detergent composition will typically comprise from 1% to about 40%, typically from 2 to 20%, or even from about 4% to about 15%, or from 5 to 10% by weight of builder(s).

The detergent composition may comprise from 1% to about 40%, typically from 2 to 20%, or even from about 4% to about 15%, or from 5 to 10% by weight of builder(s), chelant(s), or, in general, any material which will remove calcium ions from solution by, for example, sequestration, complexation, precipitation or ion exchange.

The detergent composition may comprise a chelant. Suitable chelants include diethylene triamine pentaacetate, diethylene triamine penta(methyl phosphonic acid), ethylene diamine-N′N′-disuccinic acid, ethylene diamine tetraacetate, ethylene diamine tetra(methylene phosphonic acid) and hydroxyethane di(methylene phosphonic acid). A preferred chelant is ethylene diamine-N′N′-disuccinic acid (EDDS) and/or hydroxyethane diphosphonic acid (HEDP). Preferably the ethylene diamine-N′N′-disuccinic acid is in S′S′ enantiomeric form. The composition of the invention may comprise less than 3% or less than 2% or 1% or 0.5% of each of the above mentioned chelants.

Builders include, but are not limited to, the alkali metal, ammonium and alkanolammonium salts of polyphosphates, alkali metal silicates, layered silicates, such as SKS-6 of Clariant®, alkaline earth and alkali metal carbonates, aluminosilicate builders, such as zeolite, and polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, fatty acids, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof.

The detergent composition may comprise less than 50%, in particular less than 25%, or less than 20%, 15%, 10%, or 5% by weight of phosphate and/or aluminosilicate builders.

The detergent composition may comprise from 0 to 50%, in particular from 1% to 25%, or less than 20%, or less than 15%, or less than 10%, or less than 5%, or less than 1% by weight of phosphate builder(s).

The detergent composition may comprise from 0 to 50%, in particular from 1% to 25%, or less than 20%, or less than 15%, or less than 10%, or less than 5%, or less than 1% by weight of aluminosilicate builder(s). The aluminosilicate builder may comprise zeolite.

The detergent composition may comprise from 0 to 50%, in particular from 1% to 25%, or less than 20%, or less than 15%, or less than 10%, or less than 5%, or less than 1% by weight of polycarboxylic acid(s) and salt(s) thereof.

The detergent composition may comprise from 0 to 50%, in particular from 1% to 25%, or less than 20%, or less than 15%, or less than 10%, or less than 5%, or less than 1% by weight of layered silicate(s).

The detergent compositions of the present invention may comprise from 0 to 50%, in particular from 1% to 25%, or less than 20%, or less than 15%, or less than 10%, or less than 5%, or less than 1% by weight of sodium carbonate.

The inventors have found that the composition of the invention could exhibit satisfying sudsing properties, even when a low level of builder and or of surfactant is used.

Preferably, the detergent composition comprises a dispersant. The detergent composition may comprise at least 0.2% or 0.4% or 0.6% or 0.8% or 1% by weight of dispersant, such as carboxymethyl cellulose.

Preferably, the detergent composition of the invention contains less than 3%, preferably up to 1%, and most preferably less than 0.1% or less than 0.01% or 0.001% or even 0.0001% of suds suppressor selected from the group consisting of trimethyl-, diethyl-, dipropyl-, dibutyl-, methylethyl-, phenylmethyl polysiloxane, and mixtures thereof. Preferably, the composition of the invention contain less than 3%, preferably up to 1%, and most preferably less than 0.1% or less than 0.01% or 0.001% or even 0.0001% of suds suppressor.

### Detergent Composition

The detergent composition may be in any liquid or solid form, in the form of gel, paste, dispersion, preferably a colloidal dispersion, or any combination thereof. The detergent composition is preferably in a solid form or in the form of a paste. The detergent composition may be in particulate form, for example in free-flowing particulate form. The detergent composition in solid form can be in the form of an agglomerate, granule, flake, extrudate, bar, tablet or any combination thereof.

The detergent composition may be capable of cleaning and/or softening fabric during a laundering process. The cleaning composition may be an oral care composition. The Oral care composition may be in the form of a toothpaste, dentifrice, tooth powder, tooth gel, subgingival gel, mouthrinse, denture product, mouthspray, lozenge, oral tablet, or chewing gum. The oral composition may also be incorporated onto strips or films for direct application or attachment to oral surfaces. The cleaning composition may be a hair care, or a dish care composition.

The composition may be a hand dish detergent composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

The following examples are given by way of illustration only and therefore should not be construed to limit the scope of the invention.

### EXAMPLES

### Examples 1

### Preparation and Evaluation of Cellulose Nanoparticles

Hydrophobic modification of Arbocel MF 40-100® from JRS company.

The initial contact angle of the Arbocel particles is 37°.

The particles are hydrophobically modified using fatty acid chlorides and pyridine to form esters on the cellulose hydroxyl groups. The contact angle is modified by the fatty acid chloride loading in the reaction. For higher contact angles, longer fatty acid chlorides from propanoyl (C3) chloride to decanoyl (C10) chloride are used at weight ratios of cellulose to acid chloride of 1:4. The contact angles for the derivatized particles in the target range of contact angles were between 30 and 100°. The weight ratios of cellulose to acid chloride in the reaction is chosen based on the chain length to obtain contact angles in the target range as shown in table below.

Contact angles of hydrophobically modified arbocel MF40-100® particles as a function of the ratio of cellulose to acid chloride and the chain length of the acid chloride:

| | 1:0.1 | 1:0.175 | 1:0.2 | 1:0.25 | 1:0.3 | 1:0.4 | 1:0.5 | 1:1 | 1:2 | 1:4 |
|---|---|---|---|---|---|---|---|---|---|---|
| C1 (Acetic) | | | | | | | 44 | 48 | 44 | 48 |
| C3 (Propyl) | | | | 81 | | | 97 | 98 | | |
| C6(Hexyl) | 45 | 48 | 48 | 52 | 56 | 79 | 95 | | | |
| C10 (Decyl) | 87 | | | | | | | | | |

Performance Evaluation:

Samples comprising 100 ppm of the sudsing particles of the table above, 150 ppm of LAS, 25 ppm of technical body soil in 8 gpg hardness (pH 10) water are prepared.

Suds volumes are evaluated using a cylinder method with an oscillation device.

The samples comprising the sudsing particles of the invention shows a suds boosting benefit compared to the same sample without the sudsing particles. In particular, the C1 1:4 modified particles showed an increase in foam volume of 30 mL over control (150 ppm LAS). The C3 1:0.25 modified particles showed an increase in foam volume of 20 mL. The C6 1:0.5 has the largest foam volume of any of the samples (40 mL over control).

### Example 2

### Oral Care Composition

| Ingredients | Function | 2A | 2B | 2C | 2D |
|---|---|---|---|---|---|
| Silica, dental type | Abrasive (cleaning agent) | 15.0 | 15.0 | 15.0 | 15.0 |
| NaF USP | Fluoride source for anti- | 0.243 | 0.243 | 0.243 | 0.243 |
| | caries benefit | | | | |
| Sodium dodecyl phosphate | Anionic surfactant with | | 5.0 | 5.0 | 10.0 |
| (30% soln) | functional properties | | | | |
| Zinc citrate | Antimicrobial | 0.5 | | | |
| Sodium acid pyrophosphate | Antitartar agent | 4.17 | | | |
| Sodium saccharin | Sweetener | 0.13 | 0.13 | 0.13 | 0.13 |
| NaOH (50% soln) | pH adjuster | 0.5 | 0.5 | 0.5 | 1.0 |
| CMC sodium | Thickener | 0.35 | 0.45 | 0.55 | 0.25 |
| Titanium dioxide | Opacifier | 0.25 | 0.25 | 0.25 | 0.25 |
| Carbomer 956 | Thickener | 0.30 | 0.30 | 0.30 | 0.30 |
| Flavor | Flavor | 0.80 | 0.80 | 0.80 | 0.80 |
| Sodium lauryl sulfate | Main surfactant for | 2.5 | 4.0 | 3.3 | |
| (28% soln) | Foaming | | | | |
| Cocamidopropyl Betaine | Co-surfactant for | | | | |
| (30% soln.) | foaming | | | | |
| Sorbitol solution | Humectant and Carrier | 60 | 60 | 60 | 60 |
| | (vehicle) | | | | |
| Cellulose Nano Particles (C3 | Foam Stabilizer | 2.3 | | 0.70 | |
| modified Arbocel, CA 97°) | | | | | |
| Cellulose Nano Particles (C6 | Foam Stabilizer | | | | 0.90 |
| modified Arbocel, CA 79°) | | | | | |
| Cellulose Nano Particles (C1 | Foam Stabilizer | | 0.60 | | |
| modified Arbocel, CA 48°) | | | | | |
| FD&C Blue #1 | Visual | 0.05 | 0.05 | 0.05 | 0.05 |
| USP Water | Carrier (vehicle) | Q.S | Q.S | Q.S | Q.S |

### Example 3

### Fabric Care Compositions

| | Example 3A | Example 3B | Example 3C | Example 3D |
|---|---|---|---|---|
| Component | Concentration (Weight percents) | | | |
| Sodium LAS | 12 | 14 | 8 | 5 |
| Sodium AE3S | 2 | | | |
| Sodium AE1S | | 2 | | 3 |
| Cellulose Nano Particles (C3 | 0.80 | | 8 | |
| modified Arbocel, CA 97°) | | | | |
| Cellulose Nano Particles (C6 | | | | 1.5 |
| modified Arbocel, CA 79°) | | | | |
| Cellulose Nano Particles (C1 | | 0.60 | | |
| modified Arbocel, CA 48°) | | | | |
| Cationic surfactant | 0.7 | 0.8 | 0.6 | 0.5 |
| Non ionic surfactant | 0.3 | 0.3 | | |
| Zeolite | 4 | 3 | | |
| Sodium carbonate | 32 | 25 | 30 | 34 |
| Silicate | 3 | 4 | 7 | 8 |
| Carboxy Methyl Cellulose | 0.8 | 0.7 | 0.8 | 1 |
| polymers | 11 | 10 | 14 | 13 |
| enzyme | 2 | 1.5 | 2.5 | 2.6 |
| Bleach system | 6 | 5 | 8 | 10 |
| Sodium sulfate | 15 | 24 | 12 | 15 |
| Water and miscelaneous | bal | bal | bal | bal |

Composition of examples 2A-D and 3A-D are showing good sudsing properties.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A detergent composition comprising from about 1% to about 50% by weight of anionic surfactant and from about 0.1% to about 30% by weight of sudsing particles,
wherein the sudsing particles have:
an average per number length from about 50 nm to about 50 μm,
an average per number width from about 1 nm to about 500 nm,
an average per number ratio length/width from about 1.5 to about 1000, and
an hydrophobicity measured by contact angle from about 30° to about 100°,
and wherein the weight ratio of the total amount of anionic surfactant to the total amount of sudsing particles is from about 0.05 to about 20.

2. The detergent composition according to claim 1, wherein the detergent composition comprises from about 5% to about 20% by weight of anionic surfactant.

3. The detergent composition according to claim 1, wherein the detergent composition comprises from about 0.5% to about 10% by weight of sudsing particles.

4. The detergent composition according to claim 1, wherein the weight ratio of the total amount of anionic surfactant to the total amount of sudsing particles is from about 0.1 to about 10.

5. The detergent composition according to claim 1, wherein the sudsing particles have average per number length from about 100 nm to about 900 nm.

6. The detergent composition according to claim 1, wherein the sudsing particles have average per number width from about 4 nm to about 250 nm.

7. The detergent composition according to claim 1, wherein the sudsing particles have an average per number ratio length/width from about 6 to about 250.

8. The detergent composition according to claim 1, wherein the sudsing particles have an hydrophobicity measured by contact angle from about 50° to about 90°.

9. The detergent composition according to claim 1, wherein the sudsing particles comprises a compound selected from the group consisting of polysaccharide, fatty acid, polyethylene, polypropylene, and mixtures thereof.

10. The detergent composition according to claim 1, wherein the detergent composition is selected from the group consisting of a fabric care composition, an oral care composition, and a dish care composition.

11. The detergent composition according to claim 1, further comprising a dispersing agent.

12. A detergent composition comprising from about 5% to about 20% by weight of anionic surfactant and from about 0.5% to about 10% by weight of sudsing particles,
wherein the sudsing particles have:
an average per number length from about 100 nm to about 900 nm,
an average per number width from about 4 nm to about 250 nm,
an average per number ratio length/width from about 6 to about 250, and
an hydrophobicity measured by contact angle from about 50° to about 90°,
and wherein the weight ratio of the total amount of anionic surfactant to the total amount of sudsing particles is from about 0.1 to about 10.

13. The detergent composition according to claim 12, wherein the sudsing particles comprises a compound selected from the group consisting of polysaccharide, fatty acid, polyethylene, polypropylene, and mixtures thereof.
